(19) United States
(12) Patent Application Publication
(10) Pub. No.: US 2022/0193287 A1
(43) Pub. Date: Jun. 23, 2022

(54) INTERNET OF THINGS SANITIZATION SPRAYER

(71) Applicant: Unimed Corporation, Woburn, MA (US)

(72) Inventors: Stephen Wilson, New Milford, CT (US); Samuel Dawson, Brooklyn, NY (US); Douglas Manchester, Concord, MA (US)

(73) Assignee: Unimed Corporation, Woburn, MA (US)

(21) Appl. No.: 17/174,372

(22) Filed: Feb. 12, 2021

Related U.S. Application Data
(60) Provisional application No. 63/126,557, filed on Dec. 17, 2020.

(51) Int. Cl. A61L 2/18; G06K 19/07

(52) U.S. Cl. CPC A61L 2/18; A61L 2202/16; A61L 2202/14; G06K 19/0723

(57) ABSTRACT

The instant invention relates generally to the field of disinfection of public spaces such as hospitals, schools and workplace environments.

### CROSS-REFERENCE TO RELATED APPLICATION

This application claims priority from U.S. Provisional Patent Application No. 63/126,557, filed Dec. 17, 2020 entitled INTERNET OF THINGS SANITIZATION SPRAYER which is hereby incorporated by reference herein for all purposes.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office files or records, but otherwise reserves all copyrights whatsoever.

### BACKGROUND OF THE INVENTION

### Field of the Invention

The invention relates to a spraying system with on board sensors that wirelessly connects to either a local connected device or directly to the cloud. The mobile application that is either on the spraying system or the local connected device uses the data sensors and wireless capabilities of the local connected device to capture additional data such as location, camera inputs, time, movement, operator authentication and other data and wirelessly connect with cloud based application and database.

### Description of Related Art

The advent of the novel coronavirus (COVID-19) has changed society immeasurably, and in the process, impacted technology and product development in profound ways. The research and technology communities are looking for ways to prevent and mitigate the present pandemic, minimize possible resurgence of the present pandemic, and help prevent future outbreaks and pandemics by using safe and effective sanitizing methods and fluids. Currently the most effective method to apply these has been determined to be through spray and aerosol technologies. These liquid products must be applied carefully and must be applied such that the application of the fluid is uniform and covers all of the surface consistently. In this pandemic-driven time, some companies are trying to rush the technology to market with traditional spraying technology which does not provide application and coverage information to the entity managing the asset being sanitized. While spray application can be invaluable in germicidal and sanitizing applications, industry must get the applications correct and ensure that the proper amount of sanitizer is applied to all surfaces of rooms and spaces to establish and monitor best practices. Accordingly, there is a need provide a safe and effective spraying system which can provide information as to coverage and location and the operator of any disinfection application.

### BRIEF SUMMARY OF THE INVENTION

Embodiments of the present invention can include but not limited to spraying systems such as a handheld sprayer, a backpack version, a cart based sprayer or an autonomous robot that helps eliminate human variation in spraying and mobile applications configured to interface with, but not limited to, smart phones, tablets, laptops, desktops, and/or mobile devices to allow personnel to sanitize rooms and spaces and provide useful information to the entity managing the asset being sanitized.

The present invention relates generally to the field of disinfection of public spaces such as hospitals, schools and workplace environments. Traditional methods of disinfecting involve hand spraying chemicals and mopping the surfaces by hand. An improved mechanism for spraying to combat infection is to use a “smart” Internet enabled electrostatic spraying.

The instant invention has two embodiments. The first is an electrostatic spraying system with on board intelligent controller and sensors that wirelessly connect to a client device such as an iPhone or Android mobile device running a mobile application which allows it to serve as a gateway and communicate with the spraying system and with the cloud. The mobile application uses data sensors which are integrated into the sprayer and the mobile application on the smart mobile device to communicate quantities and flow rate of disinfectant applied and geographical information such as room or space identification.

The instant invention also has a cleaning function which verifies that the electrostatic sprayer has been properly cleaned by testing the cleaning fluid until it is cleaned and does not contain any residual of the defecting liquid which could clog the electrostatic sprayer if not cleaned properly. Improper and inconsistent cleaning is the number one challenge to the reliability of existing sprayers.

The instant invention also provides a means to remind the operator to clean the electrostatic sprayer as it also has clog detection which ensures that a clogged spray nozzle does not go un-noticed. When spraying fine particles, the operator may often not even realize their sprayer is dogged and surfaces can miss being disinfected properly.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 shows the sprayer of the sanitizing system of the instant invention;

FIG. 2 shows a schematic of a spraying system of the instant invention spraying electrostatically charged particles of sanitizing disinfectant and being in communication with a smart device running on a mobile application and the mobile application being in communication with a cloud application running on a cloud computing device and the mobile application being able to read a smart tag or share its GPS location and coordinates;

FIG. 3 shows an operator applying disinfectant with the spraying system of the instant invention and the operator optionally being within a geofence;

FIG. 4 shows the sprayer with connected device and applications with connectivity to remote computer server and devices;

FIG. 5 shows the Bi-directional connection between sprayer and device via wireless or wired connection;

FIG. 6 shows the portfolio system diagram showing sprayer informatics diagram including a handheld, backpack and cart based sprayers, gateway mobile device and cloud database;

FIG. 7 is a flowchart describing the automated clog detection system of the instant invention.

FIG. 8 shows the instant invention with a laser diode which is attached to wand and provides an aiming indicator laser light beam for the operator.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

The present invention will now be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

In the following description, numerous specific details are set forth. However, it is understood that embodiments of the invention may be practiced without these specific details. In other instances, well-known structures and techniques have not been shown in detail in order not to obscure the understanding of this description. Those of ordinary skill in the art, with the included descriptions, will be able to implement appropriate functionality without undue experimentation.

References in the specification to “one embodiment” or “an embodiment,” may indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that such feature, structure, or characteristic may be deployed in connection with other embodiments whether or not explicitly described.

The terms sanitizing, sterilization sterilize and sterilizing are used interchangeably within the disclosure to mean a sanitize or sanitizing.

The terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

The term sprayer, sprayer module, spray wands and spray nozzle are used interchangeably to mean a smart sprayer that is integrated bi-directionally with a mobile gateway device to a cloud data management system through an integrated software and hardware platform.

The terms people, cleaning staff, operator, personnel, user, and individual are used interchangeably within the disclosure to mean a patient that uses the invention.

The term sanitizer refers to liquid sanitizing chemistry that is capable of being dispensed by the spraying system of the instant invention.

The term platform, system, process and method are used interchangeably within the disclosure to mean a process and utility of the invention.

All the input and display fields shown in the detail description of the invention are optional and some embodiments may use all or only some of the input fields.

The term hand sprayer, back pack sprayer, cart sprayer and autonomous robot sprayer are interchangeable and any one can replace the other and the use is intended to mean a spraying system of the instant invention.

The term communications system and communications means are used interchangeably and mean a communication method between two microprocessors, sensor devices, computers or electronic or analog devices including the associated protocol to facilitate communication.

As this invention is susceptible to embodiments of many different forms, it is intended that the present disclosure be considered as an example of the principles of the invention and not intended to limit the invention to the specific embodiments shown and described.

The prior art does not provide a system for supporting an intelligent connected spraying system. The prior art includes U.S. Pat. No. 9,914,141, entitled—Portable airless sprayer, U.S. Pat. No. 6,854,667, entitled—Spray gun having indexing air cap with quick release retaining ring, U.S. Pat. No. 9,545,643, entitled—Portable airless sprayer, U.S. Pat. No. 10,046,343, entitled—Alternate indicator for electrostatic spray gun, U.S. Pat. No. 9,610,596, entitled—alternator for electrostatic spray gun, U.S. Patent Application 20170173608, entitled—Electrostatic spray gun having external charge points, U.S. Patent Application 20040195405 entitled—Voltage and current display for electrostatic spray gun and U.S. Patent Application 20200330638 entitled—Germicidal lighting with sensor and relay for safety the contents of which are incorporated by reference in their entirety.

The instant invention is a system that can prevent disease-causing pathogens and disinfect the surfaces to stop the spread of infection causing germs and viruses. Furthermore, the instant invention is intended to be used by environmental service staff who clean and disinfectant commercial or residential living and work spaces.

Contagious illness impacts everyone in the government, correctional facilities, school, retail, commercial and office environments.

Even the best efforts at sanitation can fall short when contagious illnesses like the covid-9, cold and flu are pervasive throughout a facility. Some surfaces harboring germs simply get missed because they are complex in shape or maybe hard to reach. Surfaces such as desks and the top surface are easy to clean/disinfect, but the underside, the seat, rails, arms and legs are more difficult. There is no doubt that the places that are likely to be missed are touch points where pathogens can accumulate.

Furthermore, cleaning staff often have limited time to cover large areas where traditional spray and wiping methods take a substantial amount of time. A solution has been the use of electrostatic sprayers to disperse disinfecting solutions and liquids. Electrostatic spray disinfecting is a special type of spray system that charges disinfectant spray droplets so they are attracted to surfaces similar to opposite poles of a magnet. A battery powered electrostatic sprayer can uniformly cover vast amounts of surface area rapidly and insure coverage of all critical surfaces.

Electrostatic spraying technology improves the coverage of the disinfectant application over conventional sprayers, or misters. These conventional application devices cover only surfaces that are in the line of sight of the spray nozzle. Electrostatically applied liquids have a wrapping effect, so that complex objects and areas hidden from the line of site get coated with the sanitizing liquid. Electrostatic spraying of disinfectant solutions as part of the sanitation process can help reduce the spread of viruses such as covid-19, cold and flu.

However as one can understand that the complexity of sanitizing has increased and as good as electrostatic spraying of disinfectants is there is a need to verify that the amount of disinfectant and the location of application of the disinfectant is tracked so that the management can verify that the proper spaces have been sanitized and the proper amount of sanitizing liquid has been applied.

The instant invention relates generally to the field of disinfection of public spaces such as hospitals, schools, life sciences and workplace environments. Traditional methods of disinfecting involve hand spraying chemicals and mopping the surfaces by hand. An improved mechanism for spraying to combat infection is to use a “smart” Internet enabled electrostatic spraying system.

The instant invention solves the problem of accounting for exactly how much disinfectant as measured in milliliters per minute or milliliters per square foot (however any volume measurement that is acceptable can be used) so as to establish the best practices to disinfect an environment. The system also allows a manager to observe and adjust critical variables so that they can monitor and provide quality control with respect to operators in a work force as they use smart sprayers on established routes to apply disinfectants. The managers can verify that the proper amount of sanitizing fluid has been applied to an area based on the sanitizer used and the geo-location information recorded in the log. They can also confirm that the corners were sanitized properly by viewing the sanitizing log uploaded by the mobile application to verify that the number of corners in an area that was sanitized has been sanitized correctly. They are able to do this by viewing the geo-location information and the spraying log which captures the number of times the electrostatic power has been turned off which is indicative that the operator sanitized a corner. Turning off the electrostatic effect is vital for the sanitizing particles so they are not drawn to either wall and so they can penetrate to the inside corners where germs and pathogens often propagate This can be explained by the Faraday effect. Each route that is established can be defined and assigned by a manager to consist of any number of spraying sessions such as rooms or distinct spaces like the emergency department in a hospital or a specific hospital room that has just discharged a patient with “contact precautions” meaning they have a diagnosed infectious disease. During spraying a comprehensive log file is recorded on the sprayer and wirelessly communicated through the mobile application of the instant invention on the associated mobile device up to the cloud bases application where the data is stored. The wireless and mobile application can also be integrated into the sprayer integral microprocessor which eliminates the need for a separate mobile device. Wireless transmission can be performed by Bluetooth, Bluetooth Low Energy, ANT, ANT+, WIFI, Zigbee, other wireless connection or any similar Personal Area Networking (PAN) technology. When the wireless and mobile application is integrated into the sprayer the system it can use a wired connection such as USB, USB-A, USB-C, Lightning connector to USB, or other wired connection.

The connectivity is implemented by a module that is reusable with both hardware and software across a spraying portfolio of multiple formats that can include, but not be limited to, a handheld sprayer, a backpack version, a cart sprayer or an autonomous robot that helps eliminate human variation in spraying.

The cloud server using the cloud based application which is configured to capture uploaded data and information and to store it and display it for review and analysis utilizing either build in tools or by providing data sets which meet specified search criteria. The data from one or more sprayers of the instant invention with the associated mobile application and cloud application can store data and provide data to allow managers the ability to review and compare the information to best practices for disinfecting various spaces in greater detail and precision than currently available. This data once in the database can be viewed in a web browser in tabular form with sortable fields on such criteria as sprayer identification, operator name, time and date ranges, locations etc. The instant invention is capable of generating report on demand to meet the requirements of the users.

Current spraying methods can only estimate the usage of disinfectant over a period of time or for a certain amount of square footage. With a smart sprayer, the operator's entire workflow can be captured and reviewed and compared with best practices. The mobile application of the instant invention can be enhanced through the use of gamification techniques to increase operator compliance and productivity with positive reinforcement which is proven to influence behavior. The gamification could include providing awards such as gold, silver or bronze sanitation awards for route completion time and completeness of the sanitizing process.

The following sequence depicts typical usage:
a. Operator turns on Sprayer and logins into App;
b. Operator presses Bluetooth pairing button on the Sprayer;
c. Operator selects the Sprayer by name from the mobile application of the instant invention picklist;
d. Sprayer acknowledges a paired connection by flashing status LEDS on the spray wand;
e. Operator fills tank with disinfectant and secures the cap and puts on the backpack;
f. Operator begins spraying by pressing the wand trigger once the sprayer has fully primed with sanitizing fluid;
g. The mobile application of the instant invention receives spray flow in mL per second to record the session in the Cloud;
h. The Operator can depress the lock button on the wand for extended spraying;
i. When complete the operator can close the session on the mobile application of the instant invention and select a new session with optional comments, or capture a QR code to define the location or snap a picture of the room etc.;
j. When complete for the route or the shift the operator selects rinse cycle on the mobile application of the instant invention after replacing the tank with a tank filled with fresh water. The sprayer automatically executes a rinse cycle and records this on both the sprayer and in the Cloud. This “store and forward” system architecture and redundancy ensures data is captured even if connectivity is intermittent. Consistent maintenance after spraying corrosive chemicals is proven to extend the life of electrostatic sprayer;
k. If the operator forgets to rinse the mobile application of the instant invention will remind them until this essential step is performed;
l. A permanent record of usage is kept in non-volatile memory on the sprayer so hours on, gallons sprayed and rinse cycles capture the “mileage” on the sprayer and offer extended warranty possibilities.

The mobile application or integral microprocessor of the instant interacts wirelessly with the sprayer to perform the following functions such as:
a. Set/Get Bluetooth Low Energy (BLE), however any suitable wireless technology can be utilized and is capable of setting the discovery mode to ON or OFF;
b. Set/Get spraying function to ON or OFF;
c. Set/Get electrostatic energy to ON or OFF;
d. Get spray flow rate in ounce(milliliter)/minute;
e. Get pump current in milliamperes;
f. Get Battery Capacity in either voltage or amperes;
g. Get sprayer error codes if any and return them to the mobile application of the invention for display to the operator;
h. Get serial # and firmware Version;
i. Set/Get rinsing cycles parameters e.g., time in seconds for optimal rinse;
j. Read the on board GPS.

When utilizing the system, the first step is to turn the sprayer on and connect the mobile application of the instant invention to the sprayer wireless communication processor which is in communication with the sprayer controller. The mobile application of the instant invention then captures parameters from the sprayer so that the information can be shared to the Cloud and allows the operator to set the parameters which need to be adjusted.
a. Connected computer device(s) with sensors captures and wireless communicates with various sensors including:
   i. The location Data: which is determined by either GPS sensor, Geo-fence information, location tag information;
   ii. The camera captures QR code tags, Barcode tags and physical tags;
   iii. The On-board sensors such as LIDAR, radar, lasers, infrared and passive infrared, accelerometer;
   iv. On Board GPS sensor.
b. The system also interfaces with the onboard communication means such as Bluetooth and can receive information such as date and time and time zone from the onboard dock.
c. Connected device(s) presents mobile applications with features for operators and mangers including user interface(s), data entry, alerts, messaging and event handling.

When the sprayer has been successfully connected to the mobile application of the instant invention the mobile application of the instant invention then can either capture or allow the operator to input information into the mobile application of the instant invention such as:
a. Operator identity,
b. Fluid dispensing rate,
c. Sprayer identity,
d. Duration/Time,
e. Trigger status,
f. Pump status,
g. Electrostatic Voltage (this to be disabled to spray corners more effectively),
h. Battery level,
i. Liquid dispensed,
j. Rinse cycle information,
k. Pump information and clog status,
l. Error codes,
m. Lifetime usage statistics such as total run time in hours, minutes and seconds and volume dispensed in either gallons or liters sprayed.

Specifically, when an operator of the system is sanitizing a specific route of rooms, spaces or objects they want the mobile application of the instant invention to record their location using the geolocator data received from the GPS of the mobile device, interaction with a geofence or the system can scan a location tag if geo positioning information is not available. The location tag can be a Quick Response code (QR) code, Bluetooth tag, Zigbee tag or RFID tag. The mobile application of the instant invention provides feedback to the operator of the system that the scan was successful and to confirm that the addressed this part of the operator's route. The tag can have the information encode as to the area being sanitized or the tag can be a random identifier that is associated with the area to be sanitized in the cloud or mobile application.

Alternatively, the GPS can be integrated into the spraying system of the instant invention and the instant invention can also have the communication and processing capabilities so the communication pathway can be from the spraying system of the instant invention to the cloud application without having to have an intermediary device such as the smart device.

The sprayer system can have a number of embedded sensors to capture information and transfer the information either through a wireless or wired communication method. The system can sense fluid volume dispensed, Battery Capacity and Voltage, Pump Status and Current, Trigger Status, Electrostatic Voltage (ESV) Status, Row Rate, Chemistry Type and Dilution, Error Codes such as Clog Detection, Excess Air in the Fluid Path and Low Battery and location and movement data as well as time information. Additionally, the system can be equipped with an onboard clock to provide time stamped information and accelerometers to sense the sprayer has been dropped or damaged.

An example is when an operator is sanitizing a specific route comprised of rooms, objects or spaces by spraying sanitizer the operator's manager can see the information with respect to one or more operators on their interface device that is connected to the cloud. The cloud application can display the name and completion status of each location in an operator's route. For example: “Room 112: complete”, so that the manager knows in real time which locations have been sanitized and which locations are remaining. This is an important feature in the hotel and motel industry as it alerts the staff when they can release a room. However, if the operator is out of communication range for part of the shift the information will be uploaded to the cloud application for storage and display when a suitable connection is restored. The ability for the local storage of information on the mobile application of the invention ensures that none of my information is lost. The manager can also create assignments and task from the cloud application thereby optimizing the operator utilization.

Additionally, the system provides an automated aiming guide. This helps the operator ensure that they are aiming the spray at the objects they need to sanitize. The guide can be made from an LED or laser diode and has an optionally grid pattern. Brightness can be adjusted for daylight or low light conditions. Sanitizer coverage however is disrupted when using an electrostatic sprayer to sanitize areas with corners. Additionally, the laser aiming guide can also have a distance ranging guide that informs the operator when they are the correct distance from the target surface to spray and sanitize an object or surface.

The coverage in corners of the sanitizer is not consistent with the coverage on flat or continuous surfaces and the instant invention addresses this problem by providing a method where the operator can turn the electrostatic system (EVS) off by turning off the control located on the sprayer wand or sprayer and the sprayer can operate as a traditional sprayer to address applying the desired amount of sanitizer in a corner. The laser aiming guide provides the operator with the tolls to accomplish a complete sanitizing process by providing them the ability to turn the electrostatic sprayer and provide distance information to ensure that the sprayer is within range for the operator to sanitize the object or surface.

The EPA classifies any particle size below 40 microns as a mister/fogger and full PPE is required for the operator and often the room must be evacuated. Therefore, to make the instant invention more user friendly the sprayer of the instant invention dispenses average particle size in the range of 50 to 90 micron size particles however the preferable size is 75 micron particles. The size of particles selected has the benefit of usually not being visible beyond 3 feet and it provides the spraying system of the instant invention with an effective range of three to ten feet but preferably six feet. The particle size and range of application makes it difficult for the operator to discern coverage of the disinfectant. The instant invention solves this problem by providing a spraying system equipped with an aiming device such as a laser diode with a grid lens that shows where the spray cone from the nozzle will impact a surface six feet on average from the spray nozzle tip. This guide is designed to be safe when viewed by an operator or other individual and visible in daylight and other well-lit conditions which is the majority of the spraying use cases.

The invention can also be equipped with laser or spray activated sensors which are associated with areas to be sanitized and which are activated by the aiming device and then wirelessly communicate with the mobile application of the instant invention which verifies that a particular item or feature has been sanitized.

The instant invention also provides clog detection, error detection, routing, rinsing reminder for the operator. Clogs can result from the introduction of particulates such as dirt into the flow path of the sanitizer, clogs can develop in the flow path but again because of the small particle size this not always obvious to the operator.

The instant invention can monitor the flow rate and pump current in real time and can determine between a priming sequence and a real time nozzle clog. Priming is of the system is specified to be complete within five to thirty seconds but preferably ten seconds of the initial and sustained trigger activation. Apart from this initial time period the processor interrogates the spraying system and the flow sensor for abnormal flow rates and confirms with the spraying system pump voltage and current readings to determine if there is an obstruction (clog) in the fluid flow path between the tank and the nozzle tip of the wand. When detecting a clog, the sprayer can inform the mobile application of the instant invention which will inform the operator of the condition and provide an indicator on the spraying wand which can either flash or turn on an indicator light such as a red light to indicate a system problem. This function minimizes the issue of an operator not sanitizing an area and the problems associated with missed areas of disinfection. Clog detection also maximizes up time for the sprayer and effectiveness of the operator.

The instant invention also provides a cleaning function that verifies that the electrostatic sprayer has been properly cleaned by testing the cleaning fluid until it is cleaned and does not contain any residual of the disinfecting liquid which could clog the electrostatic sprayer if not cleaned properly. The system utilizes an LED and detector to inspect the fluid clarity and once the liquid reaches a suitable clarity, which is user definable, the system will alert the operator to discontinue cleaning. The system alternatively could measure the resistance of the cleaning fluid to indicate the cleanliness of the cleaning fluid.

The instant invention provides a means to remind and alert the operator to clean the electrostatic sprayer and it also provides for clog detection which ensures that the electrostatic sprayer does not clog and therefore the amount of disinfectant applied is the amount that was intended. The system will display if a cleaning is need based on amount of sanitizing fluid dispensed or if a clog is detected and the system can also provide indicators if the system is clean or alert the operator that a cleaning cycle is need in a specified window of time which is selectable in the mobile application set up from 10 seconds to 10 minutes.

The electrostatic sprayer connectivity to the smart mobile device application of the instant invention and the connection of the application of the instant invention to the cloud allow the application of the instant invention to determine the time spent at each location, the amount of disinfectant applied and the operator identification. This bidirectional capability provides numerous opportunities for the instant invention to collect and analyze data and provide feedback to the operators and the management to ensure that the sanitizing work is completed properly. When the operator is cleaning the system, the system checks the fluid to ensure that it is clean and then continues the cleaning cycle after detection of clean fluid for a period of time which can be selected from 1 to 180 seconds but preferably 30 seconds.

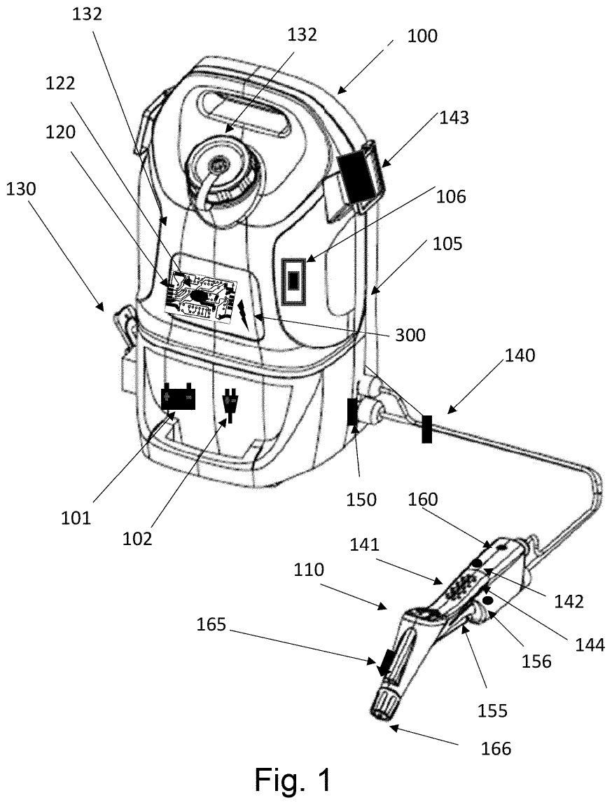

Referring now to FIG. 1- 5 and specifically FIG. 1 the sprayer of the sanitizing system of the instant invention is shown. Sprayer 100 is comprised of backpack 105 and spray wand 110 with LEDs 141, control panel 144 and ESV switch 160, Bluetooth pairing button 142, microprocessor 120, communications device 130, sanitizer sensor 140 and sanitizer dispensing sensor 150. The wand can be a removeable wand 110 having a nozzle 166 and trigger 155. Control panel 144 is capable of alerting the operator with information about the sprayer 100. Sanitizer sensor 140 measures either the clarity or the resistance of the fluid to determine if the fluid matches a set point signifying that the water is clean water when the sprayer 100 is being cleaned. The set points for said clean water is set during the set up of the sprayer. Dispensing sensor 150 equipped to monitor and measure the flow of sanitizer, voltage and amperage of the pump motor and also can measure the pressure of the fluid in the system. The flow measurement means can be a flow meter, the voltage measurement means can be a volt meter, the amperage can be measured using an amp meter. The spraying system of the instant invention can store pump run time (pump =on) in either hours, hours and minutes or minutes in non-volatile memory on sprayer which can be uploaded to the cloud application. measurement means can be an amperage meter and the pressure measurement means can be a pressure gauge or other electronic means. The set point can be entered by the operator at setup of the sprayer or set by the management or manufacturer by downloading from the cloud application to the spraying system.

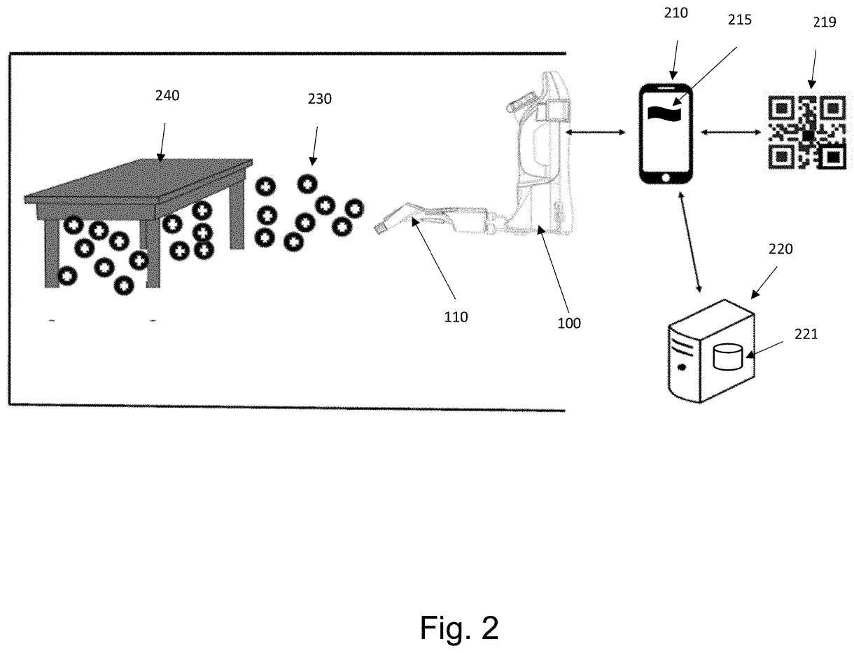

The trigger 155 can be either a mechanical release or an electronically enabled. When the trigger is electronically enabled it provides the method for the system to be engaged remotely using the commands issued from either the cloud application 221 or the mobile application 215 shown in FIG. 2.

The ESV switch 160 allows the operator to turn the electrostatic spraying system off which allows the operator to effectively sanitize hard to sanitize locations such as corners.

The spray wand 110 has laser diode 165 to assist the operate when aiming the wand when spraying sanitizer.

The sprayer 100 has batteries 101 and charging means 102 to recharge the batteries 101. The batteries are preferably Li-ion but any battery storage means can be used. The sprayer 100 can also be equipped with solar panel 143 to provide alternative charging means during operation of the sprayer 100.

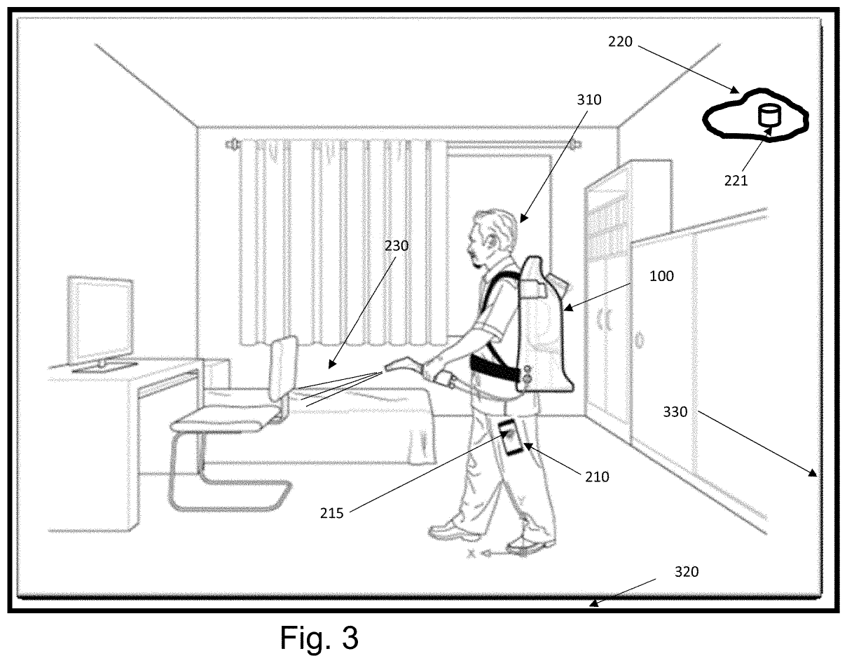

A sprayer route is comprised of one or more areas to spray which are defined by the tag 219 shown in FIG. 2, geofence 320 shown in FIG. 3 or global positioning coordinates.

Operator turns on Sprayer using switch 106 and logins into Application 215 in FIG. 2;

Operator presses Bluetooth pairing button 142 on the Sprayer 100;

Operator selects the Sprayer by name from the mobile application 215 of the instant invention picklist. Sprayer acknowledges a paired connection by flashing status LEDS 141 on the spray wand 110 control panel 144;

The operator fills tank 131 with disinfectant and secures the cap 132 and puts on the backpack;

The operator scans tag 219 using mobile application 215 shown in FIG. 2;

Operator begins spraying by pressing the wand trigger 155;

The mobile application 215 of the instant invention receives spray flow from sanitizer dispensing sensor 150 in ounces or mL per second and records the data in the session log in the Cloud;

The Operator can depress the lock button 156 on the wand for extended spraying;

When complete the operator can close the session on the mobile application 215 of the instant invention and select a new session with optional comments, or capture a new tag to define the location or take a picture of the room etc.; The picture can then be uploaded to the cloud application 221 shown inf FIG. 2 for and recorded with the session log in the cloud application 221.

When complete for the route or the shift the operator selects rinse cycle on the mobile application of the instant invention after replacing the tank with a tank filled with fresh water. The sprayer automatically executes a rinse cycle and records this. Consistent maintenance after spraying corrosive chemicals is proven to extend the life of electrostatic sprayer;

If the operator forgets to rinse the mobile application of the instant invention will remind them until this essential step is performed;

A permanent record of usage is kept in non-volatile memory 122 on the sprayer so hours on, gallons sprayed and rinse cycles capture the “mileage” on the sprayer and offer extended warranty possibilities.

Referring now to FIG. 2 the sprayer of the sanitizing system of the instant invention is shown sanitizing table 240. The sprayer 100 has spray wand 110 which sprays sanitizer particles 230. The sprayer 100 is in communication with mobile device 210 which has mobile application 215 running on it and mobile application 215 is in communication with a cloud server 220 running a cloud application 221. The system works by the operator using the mobile device 210 with mobile application 215 scanning location tag 219 which identifies the room that is being sanitized and mobile device 210 with mobile application 215 receiving quantity information from sprayer 100 and application time from the mobile device 210 and mobile application 215 sending the information including but not limited to location tag 219, quantity information from sprayer 100 and application time from the mobile device 210 to the cloud server 220 cloud application 221.

Referring to FIG. 3 which shows an operator 310 applying disinfectant 230 with the spraying system 100 which is wirelessly in communication with mobile device 210 such that mobile device 210 running mobile application 215 can collect sprayer 100 information. The operator 310 logs into the mobile application 215 and authorizes the spraying session and the mobile application 215 then collects information with respect to being within a geofence 320 which includes room 330 and the mobile device 210 running mobile application 215 reading the geofence 320 information and sending the geofence 320 information, sprayer 100 information and time information to a cloud server 220 cloud application 221. The authorization process allows the operator 310 to entered their credentials comprising of but not limited to name, identification number into mobile application 215 and mobile application 215 confirms that the operator 310 is authorized to sanitize the areas of a particular route and the route and areas to be sanitized and the coverage information need per area sanitized is confirmed to operator 310 who sets the parameters on sprayer 100. Alternatively, sprayer 100 could receive the parameters from the mobile application 215 and automatically adjust the parameters prior to commencing the spraying process.

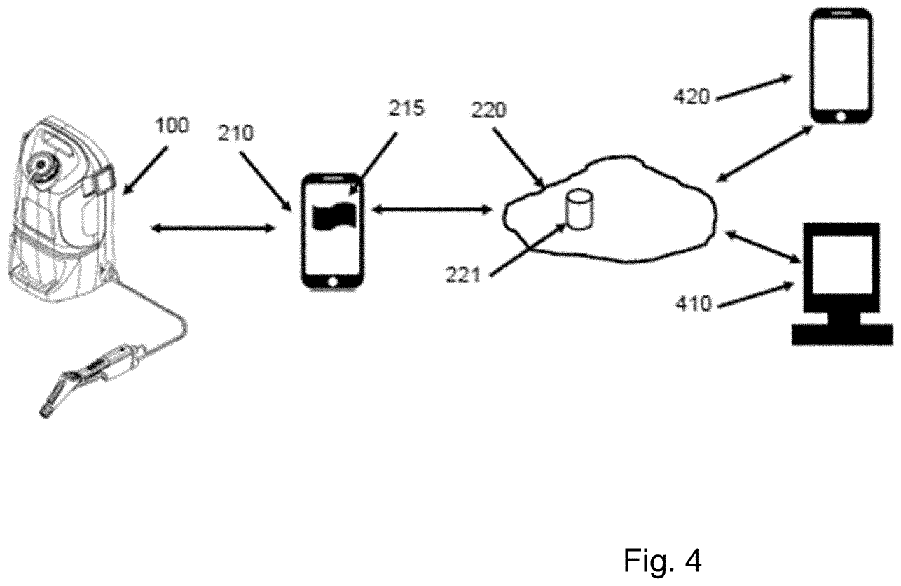

FIG. 4 shows the sprayer with connected device and applications with connectivity to remote computer server and devices. The sprayer system 100 can have a number of embedded sensors to capture information and transfer the information either through a wireless or wired communication method. The system can sense fluid volume dispensed, Battery Capacity and Voltage, Pump Status and Current, Trigger Status, Electrostatic Voltage (ESV) Status, Flow Rate, Chemistry Type and Dilution, Error Codes such as Clog Detection, Excess Air in the Fluid Path and Low Battery and location and movement of the sprayer 100 data as well as time information. Additionally, the system can be equipped with an onboard clock to provide time stamped information and accelerometers to sense the sprayer has been dropped or damaged.

The connected mobile device 210 running mobile application 215 with sensor captures and wireless communicates the information from the various sensors including:
a. The location Data: which is determined by either GPS sensor, Geo-fence information, location tag information is transfers the information to the mobile application;
b. The camera is enabled to capture any readable printed tag such as a QR code tags, barcode tags and physical tags and transfers the information to the mobile application;
c. The On-board sensors such as LIDAR, radar, lasers, infrared and passive infrared, accelerometer is read by the mobile application;
d. The mobile application also interfaces with the onboard communication means such as Bluetooth and can receive information such as date and time from the onboard clock;
e. Connected device(s) transfers the parameters read to the mobile application with features for operators and mangers including user interface(s), data entry, alerts, messaging and event handling.

The cloud server 220 cloud application 221 provide remote services and access to data and are remotely connected computer systems such as mobile device 420 and computer 410 for processing, computation storage and serving of data.

Remotely connected gateway mobile device 420 and computer 410 are connected computer device(s) which can presents mobile applications with data and features for users including route set up, assigning operators, checking certifications, user interface(s), data entry, alerts, messaging, event handling, etc.

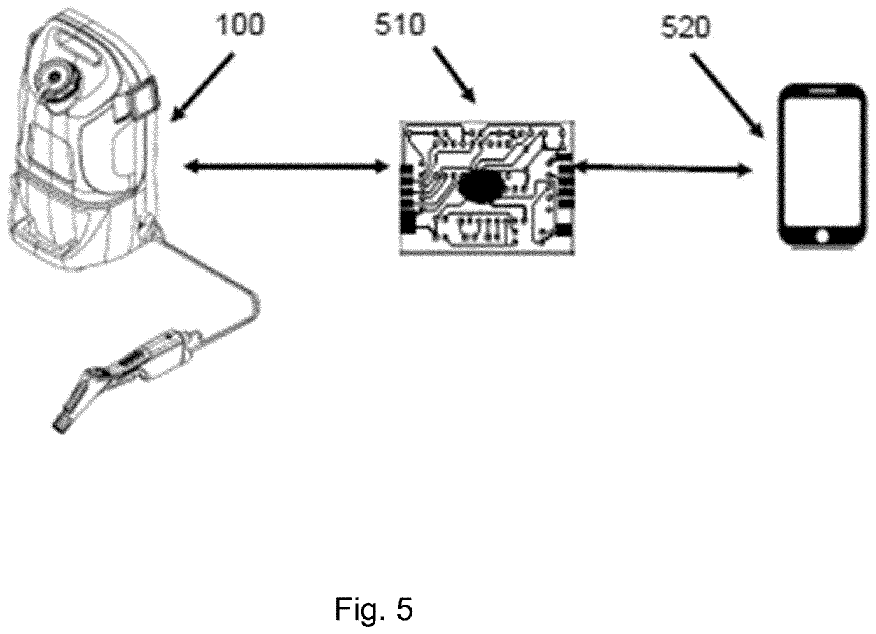

FIG. 5 shows the Bi-directional connection between sprayer and device via wireless or wired connection sprayer 100 has embedded sensors that capture and communicate using a suitable wireless or wired connection. These devices include but are not limited to battery capacity and voltage sensors, pump status and current readings, trigger status, electrostatic voltage (ESV) status, flow rate, chemistry type and dilution, error codes such as clog detection, excess air in the fluid path and low battery level.

The wireless connection 510 via Bluetooth, Bluetooth Low Energy, ANT, ANT+, WIFI or other wireless connection and wired connection via USB, USB-A, USB-C, Lightning, or other wired connection are all consistent with the intent of the communication means of the instant invention.

WIFI standards which are applicable for the system modules include but are not limited to 802.11 using frequencies such as 2.4 GHz and 5 GHz.

Connected computer device(s) 520 has access to sensors that captures and wireless communicates with other devices and various sensors including:
a. The location Data: which is determined by either GPS sensor, Geo-fence information, location tag information;
b. The camera captures picture of the area being sanitized, QR code tags, Barcode tags and physical tags;
c. The On-board sensors such as LIDAR, radar, lasers, infrared and passive infrared, accelerometer;
d. The system also interfaces with the onboard communication means such as Bluetooth and can receive information such as date and time from the onboard clock;
e. Connected device(s) presents mobile applications with features for operators and mangers including user interface(s), data entry, alerts, messaging and event handling.

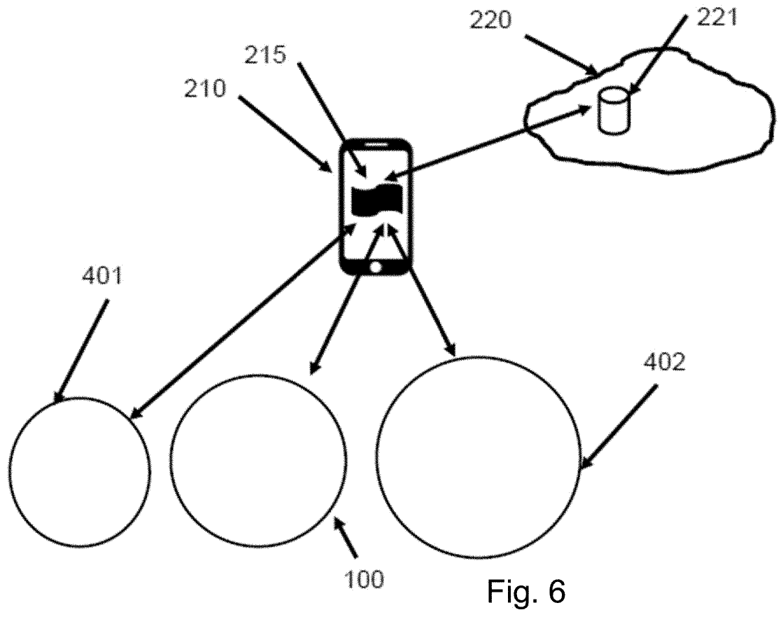

Referring to FIG. 1 through FIG. 6 and more specifically FIG. 6 is shown a portfolio system diagram showing sprayer 100 informatics diagram including sprayer 100, smart mobile device 210, mobile application 215 and cloud application 221. The Portfolio of products include hand held sprayer 401, Back pack sprayer 100 and an autonomous robot smart sprayer 402 which can all communicate with the cloud application 221. The autonomous robot smart sprayer 402 having a microprocessor in communication with a drive system to move the autonomous robot smart sprayer 402. All the sprayers have a microprocessor 120, communications device 130, sanitizer sensor 140 and sanitizer dispensing sensor 150 and have the ability to authenticate the operator 310 (not shown). The mobile application authenticates the operator 310, captures the routes and session information using either a geofence, global positioning coordinates or scanning a location tag 219 which identifies the room or areas that are being sanitized. The location tag 219 (not shown) can be any of the following Quick response code (QR) code, Bluetooth tag, Zigbee tag or RFID tag. The cloud application 221 provides the user the ability to view data in tabular form, links to various databases and the ability to create detailed reports. The hand held sprayer 401, Back pack sprayer 100 and an autonomous robot smart sprayer 402 all include an electrostatic engine to create the electrostatic spray particles, corrosion resistant nozzle 166 which is part of the spray wand 110 (not shown), batteries 101 and charging means 102, microprocessor 120, communications device 130 and associated sensors (not shown). Alternatively, the on board microprocessor can be in communication with an on board GPS device 300.

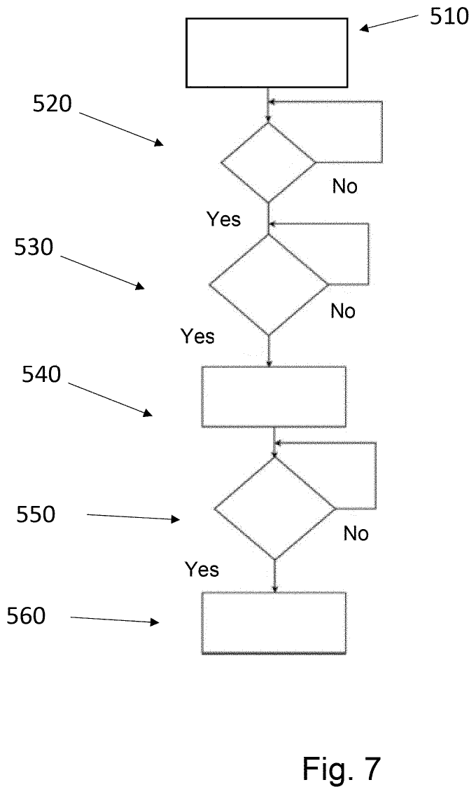

FIG. 7 is a flowchart that describe the automated clog detection system of the instant invention. In step 510 the sprayer 100 is turned on. In step 520 the sprayer 100 senses for the spray wand 110 trigger 155 to be engaged. If yes then the program proceeds to step 530 else the program waits for the trigger 155 to be engaged. In step 530 the sprayer 100 senses for the priming of the sanitizer 230 in the sprayer 100. If the sprayer 100 is being primed then the program transfers control to step 540 else the program waits for the priming of the sanitizer in the sprayer 100. In step 540 the sprayer 100 monitors the pump current and fluid pressure. In step 550 if the pump current and/or fluid pressure is higher than a set points stored in the program of the spraying systems microprocessor as measured in step 540 then the step 560 sends an alarm to the operator on the mobile application 221. The set point can be entered by the operator at setup of the sprayer or set by the management or manufacturer by downloading from the cloud application to the spraying system.

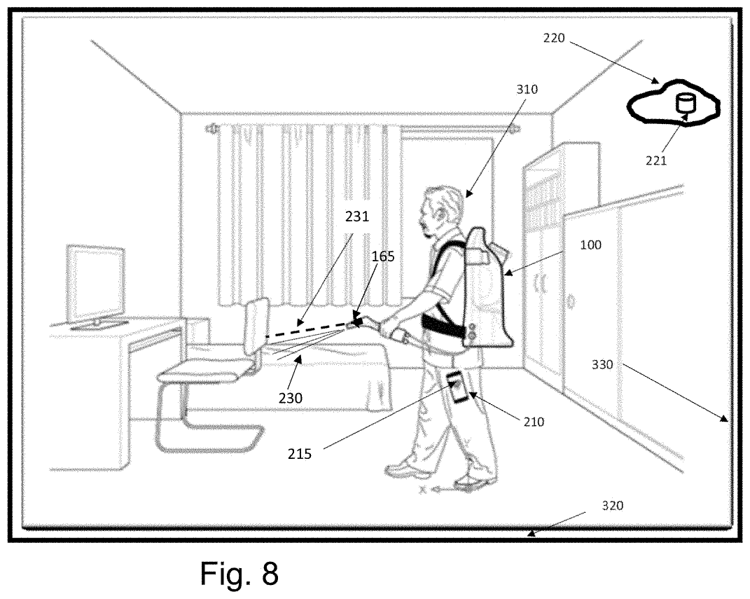

As shown in FIG. 8 laser diode 165 which is attached to wand 110 and provides an aiming indicator laser light beam 231 for the operator. The aiming indicator is helpful in corners so that the operator can turn the ESV switch 160 off allows the operator to effectively sanitize hard to sanitize locations such as corners. It also helps show the effective spray range with a focal length optimized to show the spray cone at the optimal spray distance which is typically 4 to 6′ from the nozzle 166 shown in FIG. 1.

In some embodiments, the system, method or methods described above may be executed or carried out by a computing system including a tangible computer-readable storage medium, also described herein as a storage machine, that holds machine-readable instructions executable by a logic machine such as a processor or programmable control device to provide, implement, perform, and/or enact the above described methods, processes and/or tasks. When such methods and processes are implemented, the state of the storage machine may be changed to hold different data. For example, the storage machine may include memory devices such as various hard disk drives, CD, flash drives, cloud storage, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, game controllers, video camera, camera, keyboard or gaming controller. For example, a user input may indicate a request that certain task is to be executed by the computing system, such as requesting the computing system to display any of the above described information, or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

The present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized the scope of this invention is much broader than presented by the claim. It is intended that broader claims will be submitted in an application that claims the benefit of priority from this application.

What is claimed is:

1. A sanitizing sprayer system for use by an operator capable of spraying liquid sanitizer comprising of:
a. an electrostatic sprayer having a microprocessor, a removeable tank for storing liquid sanitizer, a removeable wand having a nozzle and trigger, system sensors in communication with said microprocessor, a control panel in communication with said microprocessor;
b. a communication device;
c. said system sensors monitoring said spraying system and said microprocessor recording said system sensors monitoring said spraying system data on said microprocessor as sprayer information;
d. an area tag having area tag data defining the location or area to sanitize;
e. said microprocessor in communication with said communication device;
f. said communication device being in communication with a mobile device having a mobile application;
g. said mobile application being capable of receiving said sprayer information from said microprocessor;
h. said microprocessor having a method of detecting a clog in said electrostatic sprayer and alerting said operator of said clog.
i. Said microprocessor capable of measuring the amount of liquid sanitizer dispensed by said sanitizing sprayer system and alerting said operator and communicating amount of liquid sanitizer dispensed to said mobile application;
j. said mobile application being capable of reading said area tag and said area tag data;
k. said mobile application receiving said liquid sanitizer dispensed and associating it with said area tag data to create sanitizing area data
l. said mobile application being in communication with a cloud computer;
m. said cloud computer having a cloud application with database and said cloud application in communication with a manager managing said cloud application;
n. said mobile application in communication with said cloud application on said cloud computer;
o. said mobile application sending said sanitizing area data, said sensor data and said sprayer information to said cloud application on said cloud computer;
p. said cloud computer storing said sanitizing area data, said sensor data and said sprayer information to said cloud application.

2. The sanitizing sprayer system of claim 1 wherein said area tag is selected from the group consisting of a geofence, Quick response (QR) code, Bluetooth tag, Zigbee tag or RFID tag.

3. The sanitizing sprayer system of claim 1 wherein said microprocessor uses said method of detecting a clog comprising of sensing a trigger is turned on, said microprocessor senses that the priming of said liquid sanitizer, said microprocessor monitors the pump current, said microprocessor monitors said liquid sanitizer flow rate, said microprocessor compares said pump current and said liquid sanitizer flow rate to set points and setting a signal on said control panel or said mobile application.

4. The sanitizing sprayer system of claim 1 wherein said microprocessor uses said method of detecting clean water comprising of placing clean water in said sanitizing sprayer system spraying said clean water, said microprocessor spraying said clean water for at least one rinse cycle and said system sensors monitor said sprayer system said clean water and alerting said operator that said clean water is completed rinsing the sprayer.

5. The sanitizing sprayer system of claim 1 wherein the number of said at least one rinse cycle and volume of said clean water sprayed during said at least one rinse cycle is stored in non-volatile memory on said sanitizing sprayer.

6. The sanitizing sprayer system of claim 1 wherein said cloud application can receive instructions from a manager and communicate said instructions from said manager to said mobile application and said mobile application can communicate said instructions from a manager to said microprocessor.

7. The sanitizing sprayer system of claim 1 wherein said microprocessor can receive information from said spraying system sensor and instructions from an operator and communicate said instructions from an operator and said spraying system sensor to said mobile application and said mobile application can communicate said instructions from an operator and said spraying system sensor to said cloud application.

8. The sanitizing sprayer system of claim 1 wherein said sensors consisting of fluid volume dispensed, battery capacity, battery voltage, pump status and pump current, trigger status, electrostatic voltage (ESV) status, flow rate, chemistry type and chemistry dilution, excess air in the fluid path, accelerometers, low battery, location, said mobile application connection status, movement of said sanitizing sprayer system and time.

9. A sanitizing sprayer system for spraying liquid sanitizer comprising of:
a. an electrostatic sprayer having a microprocessor, a removeable tank for storing liquid sanitizer, a removeable wand having a nozzle and trigger, system sensors in communication with said microprocessor, a control panel in communication with said microprocessor;
b. a communication device;
c. said system sensors monitoring said spraying system;
d. said microprocessor in communication with a communication device;
e. said microprocessor in communication with a GPS device capable of receiving GPS data;
f. said microprocessor in communication with said system sensors monitoring said spraying system and said microprocessor recoding system sensors monitoring said spraying system as sprayer information;
g. said communication device being in communication with a mobile device having a mobile application;
h. said mobile application being capable of receiving said sprayer information from said microprocessor;
i. said microprocessor having a method of detecting a clog in said electrostatic sprayer and alerting said operator of said clog.
j. said microprocessor capable of timing said sanitizing sprayer system use and alerting said operator;
k. Said microprocessor capable of measuring the amount of liquid sanitizer dispensed by said sanitizing sprayer system and alerting said operator and communicating amount of liquid sanitizer dispensed to said mobile application;
l. said mobile application being capable of reading said GPS and said GPS data and associating said GPS data with an area to be sanitized and said mobile application associating said liquid sanitizer dispensed data with said GPS data to create sanitizing area data;
m. said mobile application being in communication with a cloud computer;
n. said cloud computer having a cloud application and said cloud application in communication with a manager managing said cloud application;
o. said mobile application in communication with said cloud application on said cloud computer;
p. said mobile application sending said sanitizing area data to said cloud application;
q. said cloud application sending sanitizing route information for said sanitizing area data to said mobile application;
r. said microprocessor receiving said sanitizing route information for said sanitizing area data from said mobile application and said mobile application receiving sanitizing route information for said sanitizing area data from said cloud application;
s. said microprocessor engaging said drive system and using said GPS device and said sanitizing sprayer system sanitizing said area to be sanitized;
t. said mobile application sending said sanitizing area data, said sensor data and said sprayer information to said cloud application on said cloud computer;
u. said cloud computer storing said sanitizing area data, said sensor data and said sprayer information to said cloud application.

10. The sanitizing sprayer system of claim 9 wherein said area tag is selected from the group consisting of a geofence, Quick response (QR) code, Bluetooth tag, Zigbee tag or RFID tag.

11. The sanitizing sprayer system of claim 9 wherein said microprocessor uses said method of detecting a clog comprising of sensing a trigger is turned on, said microprocessor senses that the priming of said liquid sanitizer, said microprocessor monitors the pump current, said microprocessor monitors said liquid sanitizer flow, said microprocessor compares said pump current and said liquid sanitizer flow to set points and setting a signal on said control panel.

12. The sanitizing sprayer system of claim 9 wherein said microprocessor uses said method of detecting clean water comprising of placing clean water in said sanitizing sprayer system spraying said clean water, a sensor monitors said sprayer system testing said clean water and alerting said operator that said clean water is clean.

13. The sanitizing sprayer system of claim 9 wherein said cloud application can received instructions from sanitizing sprayer system from said mobile application and said mobile application can receive said instructions to said sanitizing sprayer system said microprocessor .

14. The sanitizing sprayer system of claim 9 wherein said microprocessor can received information from said spraying system sensor and communicate said instructions from an operator to said mobile application and said mobile application can communicate said instructions from an operator to said microprocessor.

15. The sanitizing sprayer system of claim 9 wherein said sensors consisting of fluid volume dispensed, battery capacity, battery voltage, pump status and pump current, trigger status, electrostatic voltage (ESV) status, flow rate, chemistry type and chemistry dilution, excess air in the fluid path, accelerometers low battery, location, movement of the sprayer and time.

16. A sanitizing sprayer system for use by an operator capable of spraying liquid sanitizer comprising of:
a. an electrostatic sprayer having a microprocessor, a tank for liquid sanitizer and a removeable wand having a nozzle and trigger, system sensors in communication with said microprocessor, a control panel in communication with said microprocessor;
b. a communication device;
c. said system sensors monitoring said spraying system;
d. an area tag defining the area to sanitize;
e. said microprocessor in communication with said communication device;
f. said microprocessor in communication with said system sensors monitoring said spraying system;
g. said communication device being in communication with a cloud computer;
h. said cloud computer having a cloud application and said cloud application in communication with a manager managing said cloud application;
i. said microprocessor having a method of detecting a clog in said electrostatic sprayer and alerting said operator of said clog.
j. Said microprocessor capable of timing said sanitizing sprayer system use and alerting said operator;
k. said microprocessor being capable of reading sanitizing area data;
l. said microprocessor sending said sanitizing area data, said sensor data and said sprayer information to said cloud application on said cloud computer;
m. said cloud computer storing said sanitizing area data, said sensor data and said sprayer information to said cloud application.

17. The sanitizing sprayer system of claim 16 wherein said area tag is selected from the group consisting of a geofence, Quick response (QR) code, Bluetooth tag, Zigbee tag or RFID tag.

18. The sanitizing sprayer system of claim 16 wherein said microprocessor uses said method of detecting a clog comprising of sensing a trigger is turned on, said microprocessor senses that the priming of said liquid sanitizer, said microprocessor monitors the pump current, said microprocessor monitors said liquid sanitizer flow, said microprocessor compares said pump current and said liquid sanitizer flow to set points and setting a signal on said control panel.

19. The sanitizing sprayer system of claim 16 wherein said microprocessor uses said method of detecting clean water comprising of placing clean water in said sanitizing sprayer system spraying said clean water, a sensor monitors said sprayer system testing said clean water and alerting said operator that said clean water is clean.

20. The sanitizing sprayer system of claim 16 wherein said cloud application can received instructions from an operator and communicate said instructions from a manager to said microprocessor;

21. The sanitizing sprayer system of claim 16 wherein said microprocessor can received information from said spraying system sensor and communicate said information to said cloud application.

22. The sanitizing sprayer system of claim 16 wherein said sensors consisting of fluid volume dispensed, battery capacity, battery voltage, pump status and pump current, trigger status, electrostatic voltage (ESV) status, flow rate, chemistry type and chemistry dilution, excess air in the fluid path, accelerometers, low battery, location, said mobile application connection status, movement of said sanitizing sprayer system and time.

23. A sanitizing sprayer system for use by an operator capable of spraying liquid sanitizer comprising of:
a. an electrostatic sprayer having a microprocessor, a removeable tank for storing sanitizer, a removeable wand having a nozzle and trigger, system sensors in communication with said microprocessor, a control panel in communication with said microprocessor;
b. a communication device;
c. said microprocessor in communication with a GPS device and receiving GPS data;
d. said microprocessor in communication with a communication device;
e. said microprocessor in communication with said sensors monitoring said spraying system;
f. said communication device being in communication with a cloud computer;
g. said cloud computer having a cloud application and said cloud application in communication with a manager managing said cloud application;
h. said microprocessor having a method of detecting a dog in said electrostatic sprayer and alerting said operator of said dog.
i. Said microprocessor capable of timing said sanitizing sprayer system use and alerting said operator;
j. said microprocessor being capable of reading sanitizing area data;
k. said microprocessor sending said sanitizing area data, said sensor data, said GPS data and said sprayer information to said cloud application on said cloud computer;
l. said cloud computer storing said sanitizing area data, said sensor data and said sprayer information to said cloud application.

24. The sanitizing sprayer system of claim 23 wherein said area tag is selected from the group consisting of a geofence, Quick response (QR) code, Bluetooth tag, Zigbee tag or RFID tag.

25. The sanitizing sprayer system of claim 23 wherein said microprocessor uses said method of detecting a clog comprising of sensing a trigger is turned on, said microprocessor senses that the priming of said liquid sanitizer, said microprocessor monitors the pump current, said microprocessor monitors said liquid sanitizer flow, said microprocessor compares said pump current and said liquid sanitizer flow to set points and setting a signal on said control panel.

26. The sanitizing sprayer system of claim 23 wherein said microprocessor uses said method of detecting clean water comprising of placing clean water in said sanitizing sprayer system spraying said clean water, a sensor monitors said sprayer system testing said clean water and alerting said operator that said clean water is clean.

27. The sanitizing sprayer system of claim 23 wherein said cloud application can received instructions from an operator and communicate said instructions from a manager to said microprocessor;

28. The sanitizing sprayer system of claim 23 wherein said microprocessor can received information from said spraying system sensor and communicate said information to said cloud application.

29. The sanitizing sprayer system of claim 23 wherein said sensors consisting of GPS location, fluid volume dispensed, battery capacity, battery voltage, pump status and pump current, trigger status, electrostatic voltage (ESV) status, flow rate, chemistry type and chemistry dilution, excess air in the fluid path, accelerometers, low battery, location, said mobile application connection status, movement of said sanitizing sprayer system and time.
